(19) United States
(12) Patent Application Publication
(10) Pub. No.: US 2004/0248930 A1
(43) Pub. Date: Dec. 9, 2004

(54) Topical composition

(76) Inventor: Anthony Vila, Sinaloa (MX)

Correspondence Address: Catherine S Collins, Van Dyke Gardner Linn & Burkhart, 2851Charlevoix Drive SE Suite 207, PO Box 888695, Grand Rapids, MI, 49588-8695, US

(21) Appl. No.: 10/490,065

(22) PCT Filed: Sep. 18, 2002
(86) PCT No.: PCT/EP02/10522

(30) Foreign Application Priority Data
Sep. 20, 2001 (IE) S010841

(51) Int. Cl. A61K 31/4745

(52) U.S. Cl. 514/305

(57) ABSTRACT

This invention concerns the use of the brucine (2,3-dimethoxystrychnidin-10-one) or a dermatologically acceptable derivative thereof in the treatment of damaged mammalian skin.

This invention relates to a topical composition. In particular, it relates to a dermatological composition suitable for topical application to mammalian skin. Such composition is useful in the cosmetic or medical treatment of damaged mammalian skin, especially damage caused by sunburn, minor burns, ulcers or the like.

Brucine (2,3-dimethoxystrychnidin-10-one)is a highly toxic alkaloid resembling strychnine, which can be used as an addition agent for lubricants.

It has now unexpectedly been found that brucine or a dermatologically acceptable derivative thereof is useful in the regeneration of mammalian tissue.

According to the present invention there is provided the use of brucine or a dermatologically acceptable salt thereof, or a hydrate thereof, in-the manufacture of a medicament for the treatment of damaged mammalian skin.

The invention also provides a dermatological composition suitable for topical application to mammalian skin, which comprises:

(a) approximately 0.05-0.3% by weight of brucine or a dermatologically acceptable salt thereof, or a hydrate thereof;

(b) approximately 0.8-3% by weight of a hydroxy acid or a salt, ester or amide thereof;

(c) approximately 0.15-5% by weight of water; and

(d) the balance comprising a dermatologically acceptable oil or water or a combination thereof.

In the present invention, brucine is preferably used in the form of a salt, more preferably its sulphate salt. In a particularly preferred embodiment, brucine is used in the form of brucine sulphate heptahydrate. Brucine or a salt thereof or a hydrate thereof is preferably present in the composition in an amount of from approximately 0.1-0.2%, especially approximately 0.15%, by weight of the composition.

The hydroxy acid used in the composition of the present invention may be any suitable hydroxy acid, such as, for example, glycolic, lactic, malic, tartaric, citric, mandelic or salicylic acid or a mixture thereof. The hydroxy acid may be present in the form of the free acid and/or in the form of one of its associated salts, especially salts with an organic base or an alkali metal, or in the form of an ester or amide thereof. Sodium salicylate is particularly preferred. The hydroxy acid is preferably present in the composition in an amount of from approximately 1.0-2.5%, more preferably approximately 1.2-1.8%, and especially approximately 1.5%, by weight of the composition.

Component (c) of the composition comprises water in an amount of from approximately 0.15-5%, preferably from approximately 0.5-3%, more preferably from approximately 0.75-2.5%, and especially approximately 2%, by weight of the composition.

Component (d) of the composition comprises a dermatologically acceptable oil, or water, or a combination thereof. In a preferred embodiment, the composition is in the form of a water-in-oil emulsion. In a particularly preferred embodiment, component (d) consists of a dermatologically acceptable oil. Any suitable oil may be used, such as mineral oil or a vegetable oil, e.g. sunflower oil, or a mixture thereof. Pure mineral oil is preferred. In a particularly preferred embodiment, the mineral oil is free from preservative.

In a further preferred embodiment, component (d) of the composition consists of water.

The composition of the invention may also include a masking agent such as caffeine or the like, preferably in an amount of from approximately 0.03-0.08%, especially approximately 0.05%, by weight of the composition.

The composition of the invention may be formed by (a) heating the brucine or salt thereof or hydrate thereof in a suitable solvent, such as an alcohol, preferably ethanol or isopropanol, until the brucine is dissolved; (b) adding the hydroxy acid and masking agent (if used) to the water or a portion thereof and heating to a temperature in the range of from 70° C. to 85° C. until they are dissolved; (c) heating the oil, if present, to a temperature in the range of from 70° C. to 85° C.; (d) mixing the brucine and hydroxy acid solutions and any of the water remaining, or, if oil is present, adding the brucine and hydroxy acid solutions and any of the water remaining, to the heated oil; and (e) heating the resulting product to a temperature in the range of from 80° C. to 95° C. so as to evaporate substantially all of the solvent. The resulting product may then be homogenised until fully blended.

The invention will be more clearly understood from the following description thereof given by way of example only.

### EXAMPLE 1

| Ingredients | % by weight |
|---|---|
| Brucine sulphate heptahydrate | 0.15 |
| Sodium salicylate | 1.5 |
| Distilled water | 2.0 |
| Caffeine | 0.05 |
| Mineral oil (preservative-free) | qv 100 |

A water-in-oil emulsion was prepared by adding the sodium salicylate and caffeine to the water and heating to approximately 80° C. for 1-2 minutes until clear. The brucine sulphate heptahydrate was dissolved in approximately 20.0 ml of ethanol by heating to a temperature of approximately 80° C. The mineral oil was heated to approximately 80° C. and the sodium salicylate/caffeine and brucine solutions were added thereto. The resulting product was heated to approximately 90° C. for approximately 5-10 minutes to evaporate the ethanol. The product was then homogenised until fully blended.

### EXAMPLE 2

| Ingredients | % by weight |
|---|---|
| Brucine sulphate heptahydrate | 0.15 |
| Sodium salicylate | 1.5 |
| Caffeine | 0.05 |
| Distilled Water | qv 100 |

An aqueous formulation was prepared by adding the sodium salicylate and caffeine to the water and heating to approximately 80° C. for 1-2 minutes until clear. The brucine sulphate heptahydrate was dissolved in approximately 20.0 ml of ethanol by heating to a temperature of approximately 80° C. The sodium salicylate/caffeine and brucine solutions were mixed together and heated to approximately 90° C. for approximately 5-10 minutes to evaporate the ethanol.

The compositions prepared in Examples 1 and 2 were found to be effective in reducing trauma and regenerating damaged skin resulting from sunburn, minor burns and ulcers.

The results of a clinical study carried out using the emulsion of Example 1 are shown in Table I.

TABLE I

| PATIENT | | | | SEVERITY | TIME FROM | | |
|---|---|---|---|---|---|---|---|
| NUMBER/ | | | TYPE OF | OF BURN | INJURY TO | | RESULTS AND |
| GENDER | AGE | OCCUPATION | BURN | (1-5)* | TREATMENT | APPLICATION | OBSERVATIONS |
| 1. Female | 55 | Secretary | Hot Water | 3 | 1 hour | Every 5 to | Pain gone in 10 |
| | | | | | | 10 minutes | minutes. |
| | | | | | | for 3 hours | Redness gone in |
| | | | | | | | 20 minutes. |
| | | | | | | | Completely healed |
| | | | | | | | by 3^{rd} hour with |
| | | | | | | | no scarring. No |
| | | | | | | | further application |
| | | | | | | | needed |
| 2. Female | 41 | Receptionist | Curling | 2 | 30 Minutes | Every 5 to | Pain gone by |
| | | | Iron | | | 10 minutes | second application. |
| | | | | | | for 4 hours | Redness gone by |
| | | | | | | | 1^{st} hour. No |
| | | | | | | | scarring. |
| 3. Female | 39 | Lab Tech | Acid Burn | 5 | 5 Minutes | Every 10 | Pain gone by 2^{nd} |
| | | | | | | minutes for | hour. Most redness |
| | | | | | | 1 day | gone by 3^{rd} hour. |
| | | | | | | | Product applied |
| | | | | | | | throughout 1^{st} day. |
| | | | | | | | Redness greatly |
| | | | | | | | reduced on day 2. |
| | | | | | | | No pain on 2^{nd} |
| | | | | | | | day. No scarring |
| | | | | | | | noticed |
| 4. Male | 25 | Construction | Sunburn | 2 | 2 hours | Every 10 | Pain gone by 1^{st} |
| | | Worker | | | | minutes for | Hour. Redness |
| | | | | | | 1 day. Two | redacted to 75% by |
| | | | | | | applications | next morning. No |
| | | | | | | next | scarring. No visible |
| | | | | | | morning. | injury on 3^{rd} day. |
| 5. Male | 41 | Firefighter | Fire | 3 | 4 hours | Every 10 | Pain gone by 4^{th} |
| | | | | | | minutes for | application. Redness |
| | | | | | | 1 day. Once | reduced by 50% |
| | | | | | | an hour the | next morning. |
| | | | | | | next day. | Redness completely |
| | | | | | | | gone by end of |
| | | | | | | | second day. No |
| | | | | | | | pain on second |
| | | | | | | | day. No scarring |
| | | | | | | | noticed after |
| | | | | | | | one week. |
| 6. Female | 43 | Billing | Hot engine | 3 | 2 hours | Every 10 to | Most pain gone by |
| | | Agent | | | | 15 minutes | 1^{st} hour. All pain |
| | | | | | | for 1 day | gone by end of day. |
| | | | | | | | Redness gone by 4^{th} |
| | | | | | | | hour. No sign of |
| | | | | | | | scarring noticed |
| | | | | | | | after 2^{nd} day |
| 7. Male | 14 | Student | Friction | 2 | 3 hours | Every 10 | Pain gone by 1^{st} |
| | | | burn from | | | minutes for | hour. Redness cut |
| | | | rope | | | 1½ days. | in half by 4^{th} |
| | | | | | | | application. No |
| | | | | | | | scarring noticed |
| | | | | | | | one week later. |
| 8. Female | 27 | Office | Severe | 5 | 3½ days | Every 15 to | Pain reduced by |
| | | Worker | sunburn | | | 30 minutes | 50% after 4^{th} hour. |
| | | | | | | for 2 days | Pain completely |
| | | | | | | | gone by end 1^{st} day. |
| | | | | | | | Normal activities |
| | | | | | | | resumed on second |
| | | | | | | | day. Redness |
| | | | | | | | reduced by 50% by |
| | | | | | | | end of 1^{st} day. Age |
| | | | | | | | spots appeared due |
| | | | | | | | to severity of burn. |
| | | | | | | | Able to return work |
| | | | | | | | on next day. |
| | | | | | | | Completely healed |
| | | | | | | | by 4^{th} day. |

*Severity of burn indicates a standard used by those involved in this clinical study to identify the severity of a burn, 1 being least painful and 5 being most painful.
*The amount of formulation applied was sufficient to cover the area being treated.
*Patient No. 8 had previously been to three other doctors and had not found success with any treatment until she had used the product of Example 1. She had not been to work for three days.
*Patients in this clinical study were all able to return to work or school with no problems after using the product of Example 1.

The invention is not limited to the embodiments and examples described herein which may be modified or varied without departing from the scope of the invention.

1-15. (cancelled)

16. A method of treating damaged mammalian skin, the method comprising administering to a subject an effective amount of brucine (2,3-dimethoxystrychnidin-10-one)or a dermatologically acceptable salt thereof, or a hydrate thereof.

17. A method according to claim 16 wherein brucine is in the form of brucine sulphate heptahydrate.

18. A dermatological composition suitable for topical application to mammalian skin, which comprises:
(a) approximately 0.05-0.3% by weight of brucine or a dermatologically acceptable salt thereof, or a hydrate thereof;
(b) approximately 0.8-3% by weight of a hydroxy acid or a salt, ester or amide thereof;
(c) approximately 0.15-5% by weight of water; and
(d) the balance comprising a dermatologically acceptable oil or water or a combination thereof.

19. A composition according to claim 18, wherein brucine is in the form of brucine sulphate heptahydrate.

20. A composition according to claim 18, wherein brucine or a salt thereof or a hydrate thereof is present in an amount of from approximately 0.1-0.2% by weight of the composition.

21. A composition according to claim 20, wherein brucine or a salt thereof or a hydrate thereof is present in an amount of approximately 0.15% by weight of the composition.

22. A composition according to claim 18, wherein the hydroxy acid is selected from glycolic, lactic, malic, tartaric, citric, mandelic or salicylic acid, or a salt thereof or a mixture thereof.

23. A composition according to claim 22, wherein the hydroxy acid is sodium salicylate.

24. A composition according to claim 18, wherein the hydroxy acid or salt, ester or amide thereof is present in an amount of from approximately 1.0-2.5% by weight of the composition.

25. A composition according to claim 24, wherein the hydroxy acid or salt, ester or amide thereof is present in an amount of approximately 1.2-1.8% by weight of the composition.

26. A composition according to claim 24, wherein the hydroxy acid or salt, ester or amide thereof is present in an amount of approximately 1.5% by weight of the composition.

27. A composition according to claim 18, wherein component (c) of the composition comprises water in an amount of from approximately 0.5-3% by weight of the composition.

28. A composition according to claim 27, wherein component (c) of the composition comprises water in an amount of from approximately 0.75-2.5% by weight of the composition.

29. A composition according to claim 27, wherein component (c) of the composition comprises water in an amount of approximately 2% by weight of the composition.

30. A composition according to claim 18, wherein component (d) consists of water.

31. A composition according to claim 18, which is in the form of a water-in-oil emulsion.

32. A composition according to claim 31, wherein component (d) consists of a dermatologically acceptable oil.

33. A composition according to claim 18, wherein the dermatologically acceptable oil is mineral oil or a vegetable oil, or a mixture thereof.

34. A composition according to claim 33, wherein the oil is mineral oil.

35. A composition according to claim 33, wherein the mineral oil is free from preservative.

36. A composition according to claim 18, further comprising a masking agent.

37. A composition according to claim 36, wherein the masking agent is caffeine which is present in an amount of from approximately 0.03-0.08% by weight of the composition.

38. A composition according to claim 37, wherein the caffeine is present in an amount of approximately 0.05% by weight of the composition.

39. A composition according to claim 18, for use in the treatment of damaged mammalian skin.

40. A method of preparing a composition according to claim 18, comprising the following steps:
(a) heating the brucine or salt thereof or hydrate thereof in a solvent until the brucine is dissolved;
(b) adding the hydroxy acid and masking agent, if present, to the water or a portion thereof and heating to a temperature in the range of from 70° C. to 85° C. until they are dissolved;
(c) heating the oil, if present, to a temperature in the range of from 70° C. to 85° C.;
(d) mixing the brucine and hydroxy acid solutions and any of the water remaining, or, if oil is present, adding the brucine and hydroxy acid solutions and any of the water remaining, to the heated oil; and
(e) heating the resulting product to a temperature in the range of from 80° C. to 95° C. so as to evaporate substantially all of the solvent.

41. A method according to claim 40, wherein the solvent in step (a) is ethanol.
